(19) United States
(12) Patent Application Publication
(10) Pub. No.: US 2005/0038394 A1
(43) Pub. Date: Feb. 17, 2005

(54) Syringe with re-use inhibiting mechanism

(76) Inventor: Jorg Schwarzbich, Bielefeld (DE)

Correspondence Address: RICHARD M. GOLDBERG, 25 EAST SALEM SREEET, SUITE 419, HACKENSACK, NJ, 07601, US

(21) Appl. No.: 10/915,111

(22) Filed: Aug. 10, 2004

(30) Foreign Application Priority Data
Aug. 16, 2003 (DE) 203 12 677.7

(51) Int. Cl. A61M 5/32

(52) U.S. Cl. 604/218

(57) ABSTRACT

A syringe includes a piston (12) and a cylinder (11) having a cone (20) at one end thereof, and a re-use inhibiting mechanism having a trigger element (30) formed at the piston (12), wherein the trigger element (30) is provided at the front end of the piston (12) such that it cooperates with the cone (20) of the cylinder (11) when the piston (12) is pushed into the cylinder.

The invention relates to a syringe comprising a piston and a cylinder provided with a cone at its front end, and having a re-use inhibiting mechanism comprising a trigger element formed at the piston.

The term re-use inhibiting mechanism as used in this invention includes also a mechanism in which the re-use is not prevented by active locking or blocking, but by making a component inoperative, that is essential for the function of the syringe.

The re-use of used syringes may lead to the spreading of serious diseases such as aids or hepatitis. The British patent application GB 2 315 414 A discloses a disposable syringe according to the preamble of claim 1 having a re-use inhibitor. The syringe comprises in its cylinder an annular member which is either a groove in the internal wall of the cylinder or a bead. The piston rod has barbs in the form of lateral flaps which permit the piston to be pushed into the syringe but engage in the groove-or at the bead when the piston rod is withdrawn, and thereby block the piston rod. Thus, the piston of a syringe that has once been used, may no longer be withdrawn far enough for using the syringe another time. However, it is a drawback that the syringe must have an undercut in the interior of the cyinder, i.e. either the groove or the portion between the cone and the bead, and is therefore difficult to withdraw from a mold.

It is an object of the invention to provide a syringe of the type indicated above, the cylinder of which has a simpler construction and can be manufactured more easily.

According to the invention, this object is achieved by the feature that the trigger element is formed that the front end of the piston such that it cooperates with the cone of the cylinder when the piston is pushed into the cylinder.

In the syringe according to the invention, the trigger element for the re-use inhibition is simply actuated by being inserted into the cone, e.g. the so-called Luer-cone, which is present at the syringe, anyway. As a result, any undercuts at the cylinder of the syringe are no longer necessary. If desired, counter-elements which cooperate with the trigger element, may be formed at the free end or in the interior of the cone. However, these counter-elements may also be manufactured without undercut, thanks to their position at the end or at the tapering internal wall of the cone.

The syringe with the above construction has several advantages. The cylinder can easily be manufactured because it has an essentially uniform internal cross-section without any grooves or beads. In comparison to a conventional disposable syringe without re-use inhibition, only a minor modification, if any, at the cone is necessary. Thus, the cylinder has a very simple construction. As another advantage, the cylinder may have a uniform wall thickness. A great advantage is that the trigger element utilizes the space present in the cone, so that the length of the cylinder hardly needs to be enlarged in comparison to a conventinal disposable syringe without re-use inhibition. Another advantage is that the smooth internal walls of the cylinder permit a smooth movement of the piston during the injection process and, as the case may be, permit also to dispense with special piston seals.

Useful embodiments of the invention are indicated in the dependent claims.

In a first embodiment, the trigger element is formed by at least one elastic tongue at the front end of the piston, said tongue having a barb which, when it engages behind a step formed at the internal side of the cone, locks the piston against retraction. Thus, a re-use of the syringe is inhibited. Here, the length of the cylinder need not be larger than in a conventional disposable syringe, since the tongue utilizes the space present within the cone.

When the cylinder is manufactured by injection molding, the mold portion used for forming the step can easily be withdrawn from the front end of the cone.

The syringe may have a break-off point between the piston and a piston rod. If the piston rod is abusively withdrawn after the piston has been locked, the piston rod tears off behind the piston. Since the piston remains within the cylinder, a re-use of the syringe is still inhibited.

In a second embodiment, the syringe has, as an alternative or in addition to the trigger mechanism described above, an annular piston seal which is held at the piston by a bracket which is released when the trigger element slides onto the cone. When the bracket is released, the piston seal is not entrained by the piston, so that no subatmospheric pressure can be built up in the syringe when the piston rod is withdrawn. A re-use of the syringe is thereby inhibited. Another advantage is that it is possible to use a conventional syringe body without any moficiations.

Preferably, the piston seal is biased elastically and expands axially when the bracket is released. At that instant, the piston seal may deform in such a manner that it is no longer caught by the bracket when, subsequently, the piston rod is withdrawn and the bracket is returned, for example, into its original position.

As an alternative or additionally, the resilient element is adapted to be locked in a position in which the bracket is released. Due to the resilient element being locked in this way, the piston seal is not caught by the bracket which is still in the released state, and is not entrained by the piston when the latter is withdrawn.

Preferably, the bracket is formed by a disk-shaped part of the piston and at least one radial projection at the trigger element, which hold the piston seal under an axial bias. The bracket can easily be released due to the fact that, when the trigger element slides onto the cone, the radial projection is deflected towards the central axis of the syringe to such an extent that the piston seal is no longer caught and expands axially. Then, as soon as the piston is drawn away from the piston seal by withdrawing the piston rod, the syringe is no longer sealed tight, so that a subatmospheric pressure may no longer be built up in the syringe. In addition, this permits a particularly simple construction of the piston.

Preferably, the piston seal has an internal bead which is more rigid than the outer part of the seal. The more rigid internal bead may for example be held by relatively small radial projections of the bracket, whereas the piston seal may be supported on the side of the piston by a larger area. By appropriately adapting the rigidity of the internal bead, the position of the piston in which the bracket releases the internal bead, may be suitably determined.

Embodiment examples of the invention will now be explained in conjunction with the drawings, wherein:

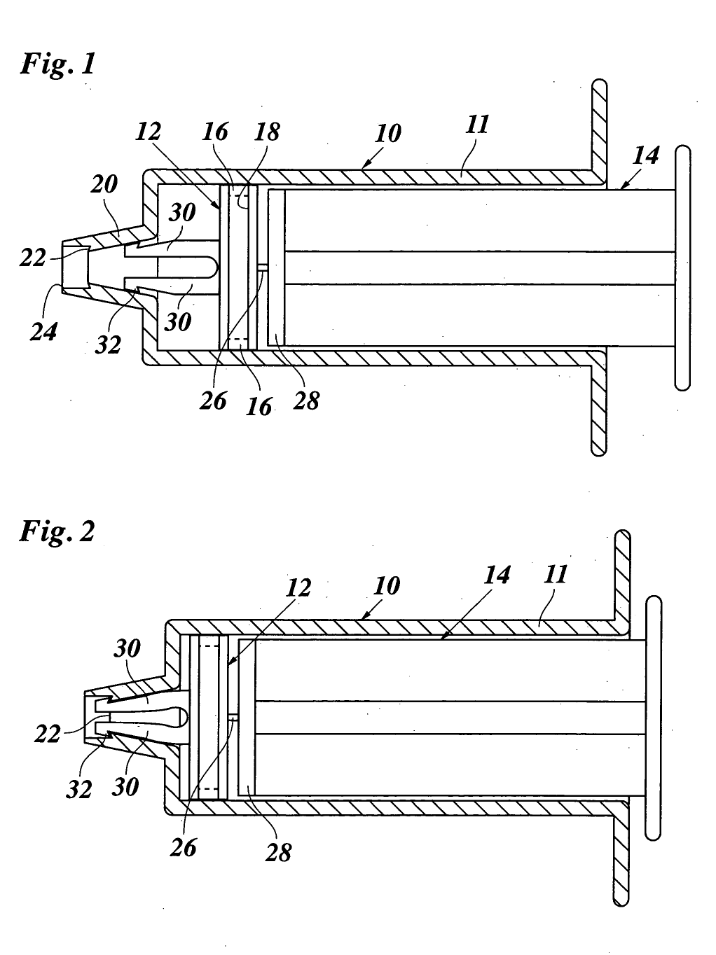

FIG. 1 shows a first embodiment of a syringe the piston of which has tongues with barbs:

FIG. 2 shows the syringe of FIG. 1 with the piston in the locked state,

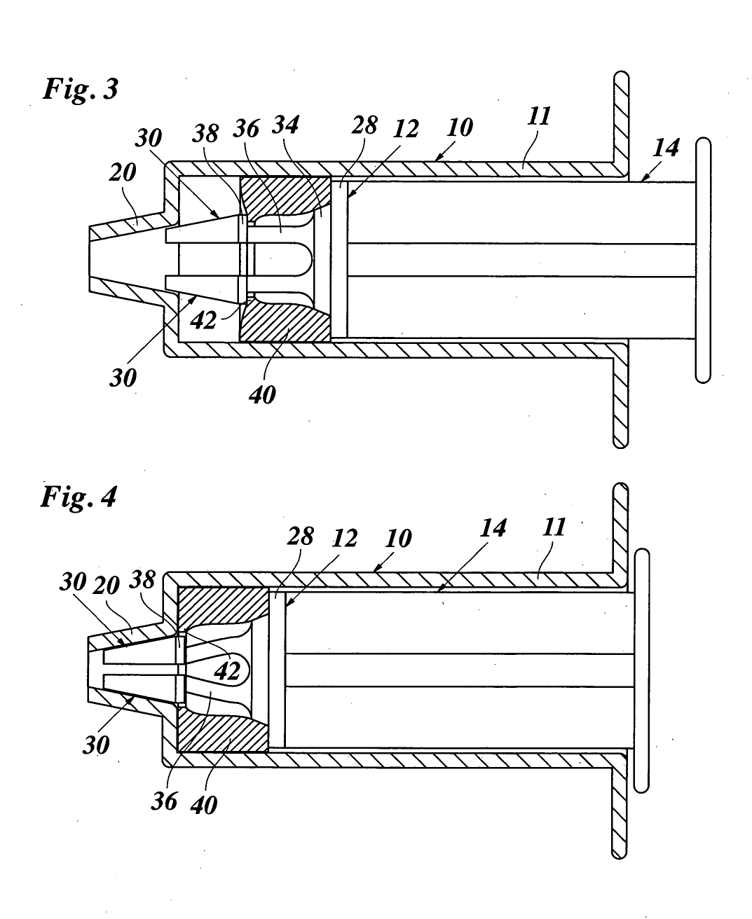

FIG. 3 shows a second embodiment of a syringe having a piston seal held in a bracket;

FIG. 4 shows the syringe of FIG. 3 with the bracket in the released state;

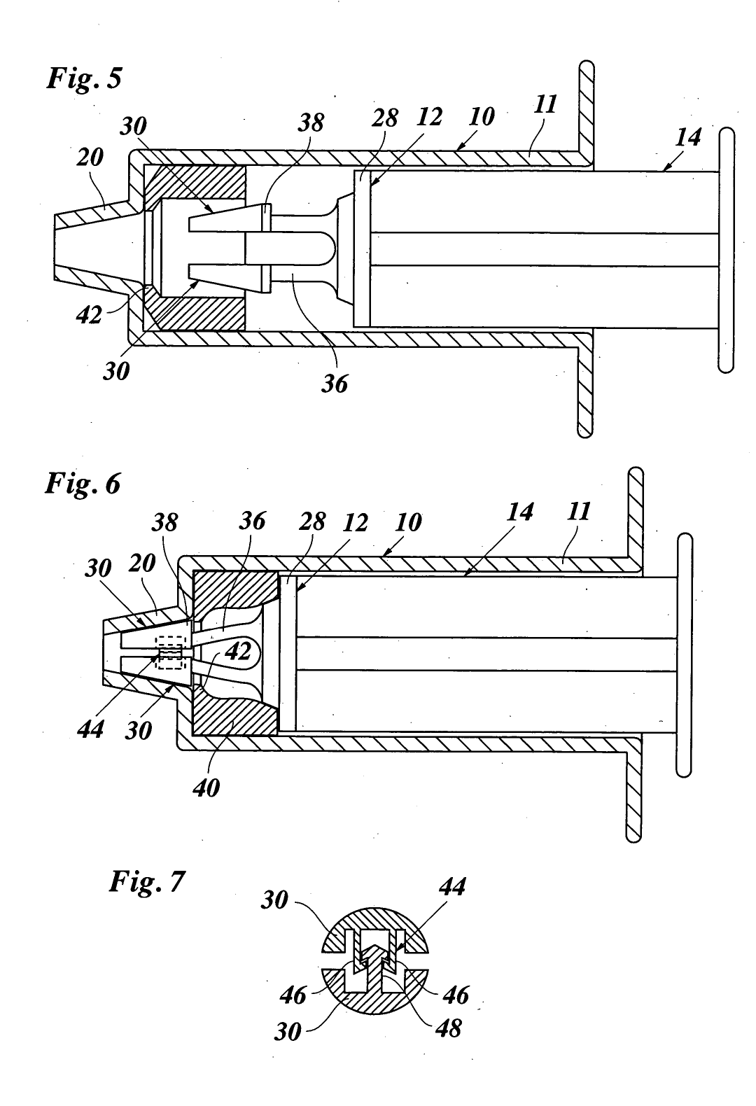

FIG. 5 shows the syringe of FIG. 4 with the piston being withdrawn;

FIG. 6 shows a modification of the syringe of FIG. 4, comprising a locking member for locking the tongues at one another; and

FIG. 7 shows a cross-section of the tongues locked at one another.

With the exception of FIG. 7, all drawing figures are longitudinal sections, wherein, however, the piston has not been shown in section.

The syringe shown in FIGS. 1 and 2 consists of two components: a body 10 forming a cylinder 11, a piston 12 having a piston rod 14, and a piston seal 16 which is held in an annular groove 18 of the piston 12. The body 10 and the piston 12 including the piston rod 14 are made of synthetic resin. The piston seal 16 may be made of rubber but may also be made of synthetic resin or formed integrally with the piston 12.

The body 10 has at its front end a cannula socket shaped as a cone 20. In the vicinity of its tip, the cone 20 has an annular step 22 from which the cone 20 extends to an opening 24, at the front end with constant internal radius.

Through a web 26 which forms a brake-off point, the piston 12 is connected to a disk 28 which closes-off the front end of the piston rod 14. The piston 14 has a cross-shaped cross-section. Its outer radius is smaller than or equal to the internal radius of the cylinder 11.

On its front side, the piston 12 has two resilient trigger elements in the form of elastic tongues 30, the front ends of which are shaped such that they may enter into the cone 20 and thereby slide upon the cone 20. A barb 32 is formed at each of the tongues 30 and engages behind the step 22, which is formed complementary thereto, when the tongues are thrust further into the cone 20, so that a re-use inhibition is triggered.

FIG. 2 illustrates the situation after the barbs 32 have engaged behind the step 22 and the elastic tongues 30 have flexed back. The piston 12 is thereby locked against withdrawal. If, nevertheless, the piston rod 14 is drawn back with larger effort, it is torn off from the piston 20 at the web 26. In any case, the piston 12 is locked against withdrawal by the barbs 32 engaging behind the step 22, so that the piston is not operative for a renewed use of the syringe.

A possible modification of this embodiment may be that, in reverse to the principle described above, one or more elstic tongues are molded to the internal surface of the cone, and the trigger element at the piston forms a step at which the tongues are lokked. Even in this case the tongues may be manufactured without undercut, due to the taper of the internal surface of the cone.

In a further modification, the trigger element at the piston may be made essentially rigid and may cooperate with a complementary contour at the cone in press button fashion. In this case, the user will feel a detent immediately before the re-use inhibition becomes active.

FIGS. 3, 4 and 5 show a second embodiment. The body 10 has no step at the cone 20 but corresponds to the body of a conventional syringe. At the piston 12, the tongues 30 are formed on a socket 34 on the disk 28. The tongues have a reduced portion 36 with radial projections 38 being formed at the front end thereof. From these projections, the tongues 30 taper towards their tip and are so shaped that they may be inserted into the cone 20 up to approximately the front end of the reduced portion 36.

The radial projections 38 together with the disk 28 form a bracket for an annular piston seal 40 which is supported with one of its ends on the disk 20 and has at its other end an internal bead 42 supported at the radial projections 38 of the tongues 30 and thus seals the disk 28 of the piston against the cylinder 11 of the body 10. The piston seal 40 is made of an elastic material, e.g. rubber or a suitable polymer. The portion of the piston seal 40 forming the internal bead 42 has a greater rigidity than the external portion. This assures that the piston seal 40, which is axially biased in the bracket, can be held by the radial projections 38.

When the piston 12 is thrust into the body 10 by means of the piston rod 14, the tongues 30 slide upon the cone 20 and are deflected inwardly. In the positioin shown in FIG. 4, the tongues 30 have been deflected inwardly so far that the radial projections 38 no longer catch the internal bead 42 of the piston seal 40 which is thereby released from the bracket. At that instant, the elastic piston seal 40 expands in axial direction, so that the internal bead 42 engages the end face of the body 10. The piston 12 may be thrust into the body 10 slightly further, with axial compression of the piston seal 40. In any case, when the piston 12 is withdrawn, the radial projections 38 of the tongues 30 will no longer catch the internal bead 42 of the piston seal 40. The piston seal 40 will therefore remain in the front part of the body 10, as can be seen in FIG. 5.

The outer radius of the disk 28 of the piston 12 is smaller than the inner radius of the cylinder 11 of the body 10. Releasing the bracket for the piston seal 40 through expansion of the elastic piston seal 40 has therefore the effect that the piston seal 40 no longer seals the piston 12 against the cylinder 11 of the body 10 when the piston 12 is withdrawn. The piston has thereby been made inoperative for a renewed use of the syringe. In FIG. 5, the piston seal 40 is shown in its axially fully expanded position.

FIG. 6 shows a variant of the second embodiment, in which the bracket has been released by locking the tongues 30 against one another, after they have been approached to one another sufficiently far, by means of a locking device 44, in a position in which the radial projections 38 can freely pass through the internal bead 42 of the piston seal 40 when the piston 12 is withdrawn. This variant therefore imposes no strict requirements on the manufacturing tolerances of the internal bead 42, because, independently of the inner radius, the bracket will in any case be released when the tongues 30 are locked against one another.

The locking device 44, which has been shown in an enlarged cross-section in FIG. 7, comprises, on one tongue 30, two elastic locking hooks 46 which snap-in at a counterpart 48 of the other tongue 30.

Of course, the locking device 44 may also be combined with the function principle described in conjunction with FIGS. 3 to 5. In this case, the axial expansion of the piston seal and the locking of the tongues 30 provide a mechanism with two systems which operate independently from one another and each of which assures that the bracket for the piston seal 40 is released and the piston is thereby made inoperative for a renewed use of the syringe.

1. A syringe comprising:
a piston,
a cylinder having a cone at one end thereof, and a re-use inhibiting mechanism comprising a trigger element formed at a front end of the piston, such that the trigger element cooperates with the cone of the cylinder when the piston is pushed into the cylinder.

2. The syringe of claim 1, wherein the cone includes a step and the trigger element is formed by at least one elastic tongue having a barb which, when engaging behind the step in the cones, locks the piston against retraction.

3. The syringe of claim 2, further comprising a piston rod and a break-off point between the piston and the piston rod.

4. The syringe of claim 1, further comprising a piston seal held at the piston by a bracket which is released when the trigger element slides upon the cone.

5. The syringe of claim 4, wherein the piston seal is elastic and is biased such that the piston seal expands axially when the bracket is released.

6. The syringe of claim 4, further comprising a locking device for locking the trigger element in a position in which the bracket is released.

7. The syringe of claim 4, wherein the bracket is formed by a member of the piston and at least one radial projection at the trigger element, and axially clamps the piston seal.

8. The syringe of claim 4, wherein the piston seal has an internal head that is more rigid than an outer portion of the piston seal.
